(19) United States
(12) Patent Application Publication
(10) Pub. No.: US 2025/0273341 A1
(43) Pub. Date: Aug. 28, 2025

(54) PREDICTING FATIGUE AND INJURY RISK USING DIGITAL TWIN OF USER

(71) Applicant: Texas State University, San Marcos, TX (US)

(72) Inventors: Abhimanyu Sharotry, San Marcos, TX (US); Jesus Jimenez, New Braunfels, TX (US); Francis A. Méndez Mediavilla, San Marcos, TX (US)

(21) Appl. No.: 19/063,165

(22) Filed: Feb. 25, 2025

Related U.S. Application Data
(60) Provisional application No. 63/557,693, filed on Feb. 26, 2024.

(51) Int. Cl. G16H 50/30; A61B 5/00; A61B 5/11; A61B 5/397; G06V 10/82; G06V 40/20

(52) U.S. Cl. CPC G16H 50/30; A61B 5/1118; A61B 5/1126; A61B 5/397; A61B 5/486; A61B 5/7264; A61B 5/7275; A61B 2503/20; A61B 2562/0219; G06V 10/82; G06V 40/20

(57) ABSTRACT

A computer-implemented method, system, and computer program product for predicting fatigue and injury risk for users, such as workers performing material handling operations, in real-time. Motions performed by a user, such as lifting, carrying, and manipulating objects, are captured in real-time, such as via camera-based optical devices, electromyography sensors, and inertia measurement units. Upon capturing motions performed by the user, such captured motions are utilized by a trained machine learning model to predict fatigue and injury risk to users. Such a prediction is made by the trained machine learning model by comparing the captured motions to predefined thresholds of range of motion constraints based on the biomechanical parameters of the user from a digital twin model of the user. Feedback may then be provided based on the predicted fatigue and injury risk of the user, such as in the form of video, audio, and/or haptic alerts.

### TECHNICAL FIELD

The present disclosure relates generally to safety monitoring tools, and more particularly to predicting fatigue and injury risk to users, such as workers performing material handling operations, using a digital twin (digital model) of the user.

### BACKGROUND

Workers within industrial sectors, such as manufacturing, warehousing, shipping and logistics, construction, and mining, face numerous hazards during daily operations. Globally, tens of millions of workers in manually intensive jobs experience musculoskeletal disorders and injuries from occupational hazards, which causes billions in healthcare costs annually. Specifically, in manufacturing, ergonomic hazards for assembly workers who perform repetitive motions over long hours are of particular concern and can lead to overuse injuries. In warehousing and shipping, order fulfillment roles involve substantial manual material handling, lifting, bending, and twisting motions that strain the body and cause frequent sprains and falls. In construction and mining, workers are exposed to especially significant safety risks while working with heavy machinery and in dangerous environments.

There is a substantial need and incentive for companies in these industries to invest in safety monitoring tools to protect their workers.

As a result, there have been attempts to improve workplace safety and ergonomics by detecting potentially harmful motions or excessive fatigue. For example, methods, such as standard questionnaires after completing a job, have been used in the past to analyze fatigue in construction workers. Unfortunately, relying on such questionnaires only addresses fatigue and injuries after such occurrences.

Various ergonomic assessment tools, such as Rapid Upper Limb Assessment (RULA) and the job strain index method, are commonly practiced in industries to identify repetitive movements. In order to identify strained postures, observational tools, such as Rapid Entire Body Assessment (REBA) and the Ovako Working Assessment System (OWAS), provide feedback based on an experienced user's scoring system. The National Institute for Occupational Safety and Health (NIOSH) lifting equation, Snook tables, and Liberty Mutual tables provide information on safe load capacity. The commonly used Borg scale assess fatigue by subjective worker feedback.

Such methods require post-experiment evaluation resulting in an inability to provide real-time feedback to the operator. Furthermore, another limitation of currently used methods is not including the object's weight, a significant component of manual material handling (MMH) tasks, as a decision variable.

In recent times, virtual human factor (VHF) tools such as virtual reality, digital human models, and discrete event simulation allow the user to perform an ergonomic assessment to systems not yet constructed. However, such tools posses the limitation of being used only in the design stage of the process. Furthermore, such tools may be limited to light assembly work and only considering loads at the shoulder joint.

Unfortunately, such current tools are deficient in predicting fatigue and injury risk for users, such as workers performing material handling operations, in real-time.

### SUMMARY

In one embodiment of the present disclosure, a computer-implemented method for predicting fatigue and injury risk to users comprises capturing motions performed by a user. The method further comprises predicting fatigue and injury risk of the user using a trained machine learning model by comparing the captured motions to predefined thresholds of range of motion constraints based on biomechanical parameters of the user from a digital twin model of the user. The method additionally comprises providing feedback based on the predicted fatigue and injury risk of the user.

Other forms of the embodiment of the computer-implemented method described above are in a system and in a computer program product.

The foregoing has outlined rather generally the features and technical advantages of one or more embodiments of the present disclosure in order that the detailed description of the present disclosure that follows may be better understood. Additional features and advantages of the present disclosure will be described hereinafter which may form the subject of the claims of the present disclosure.

### BRIEF DESCRIPTION OF THE DRAWINGS

A better understanding of the present disclosure can be obtained when the following detailed description is considered in conjunction with the following drawings, in which:

FIG. 1 illustrates an embodiment of the present disclosure of a system for practicing the principles of the present disclosure;

FIG. 2 is a diagram of the software components used by the fatigue and injury risk prevention mechanism for predicting fatigue and injury risk to users in accordance with an embodiment of the present disclosure.

FIG. 3 illustrates an embodiment of the present disclosure of the hardware configuration of the fatigue and injury risk prevention mechanism which is representative of a hardware environment for practicing the present disclosure;

FIG. 4 is a flowchart of a method for training a machine learning model to predict fatigue and injury risk for users in real-time in accordance with an embodiment of the present disclosure; and

FIG. 5 is a flowchart of a method for predicting fatigue and injury risk for users in real-time in accordance with an embodiment of the present disclosure.

### DETAILED DESCRIPTION

As stated above, workers within industrial sectors, such as manufacturing, warehousing, shipping and logistics, construction, and mining, face numerous hazards during daily operations. Globally, tens of millions of workers in manually intensive jobs experience musculoskeletal disorders and injuries from occupational hazards, which causes billions in healthcare costs annually. Specifically, in manufacturing, ergonomic hazards for assembly workers who perform repetitive motions over long hours are of particular concern and can lead to overuse injuries. In warehousing and shipping, order fulfillment roles involve substantial manual material handling, lifting, bending, and twisting motions that strain the body and cause frequent sprains and falls. In construction and mining, workers are exposed to especially significant safety risks while working with heavy machinery and in dangerous environments.

There is a substantial need and incentive for companies in these industries to invest in safety monitoring tools to protect their workers.

As a result, there have been attempts to improve workplace safety and ergonomics by detecting potentially harmful motions or excessive fatigue. For example, methods, such as standard questionnaires after completing a job, have been used in the past to analyze fatigue in construction workers. Unfortunately, relying on such questionnaires only addresses fatigue and injuries after such occurrences.

Various ergonomic assessment tools, such as Rapid Upper Limb Assessment (RULA) and the job strain index method, are commonly practiced in industries to identify repetitive movements. In order to identify strained postures, observational tools, such as Rapid Entire Body Assessment (REBA) and the Ovako Working Assessment System (OWAS), provide feedback based on an experienced user's scoring system. The National Institute for Occupational Safety and Health (NIOSH) lifting equation, Snook tables, and Liberty Mutual tables provide information on safe load capacity. The commonly used Borg scale assess fatigue by subjective worker feedback.

Such methods require post-experiment evaluation resulting in an inability to provide real-time feedback to the operator. Furthermore, another limitation of currently used methods is not including the object's weight, a significant component of manual material handling (MMH) tasks, as a decision variable.

In recent times, virtual human factor (VHF) tools such as virtual reality, digital human models, and discrete event simulation allow the user to perform an ergonomic assessment to systems not yet constructed. However, such tools posses the limitation of being used only in the design stage of the process. Furthermore, such tools may be limited to light assembly work and only considering loads at the shoulder joint.

Unfortunately, such current tools are deficient in predicting fatigue and injury risk for users, such as workers performing material handling operations, in real-time.

The embodiments of the present disclosure provide a means for predicting fatigue and injury risk for users, such as workers performing material handling operations, in real-time by capturing motions performed by the user, such as in real-time. For example, the motions performed by the user are captured using a camera-based optical tracking device. In another example, the motions performed by the user are captured using an inertia measurement unit, which may be worn by the user, such as on the user's wrist. In a further example, the motions performed by the user are captured by an electromyography sensor worn by the user. Upon capturing motions performed by the user, such captured motions are utilized by a trained machine learning model to predict fatigue and injury risk to users. In one embodiment, such a prediction is made by the trained machine learning model by comparing the captured motions to predefined thresholds of range of motion constraints based on biomechanical parameters of the user from a digital twin model of the user. Range of motion constraints, as used herein, refer to the limitations or restrictions placed on the movement of a joint or body part, defining the maximum extent to which it can move in a specific direction, in order to prevent fatigue or injury risk. Biomechanical parameters, as used herein, refer to characteristics relating to the function and motion of the mechanical aspects of users. A digital twin model, as used herein, refers to a digital model of the user that serves as the effectively indistinguishable digital counterpart of the user for practical purposes, such as simulation, testing, and monitoring. In one embodiment, predefined thresholds for such motion constraints, which may be established by experts based on biomechanical parameters of the user from a digital twin model of the user, are used to determine if the user is subject to a risk of fatigue and/or injury risk. For example, feedback concerning the user being subject to the risk of fatigue and/or injury risk may be provided in response to a captured motion (right lateral flexion of lumbar spine at) 25° exceeding a predefined threshold of a range of motion constraint (right lateral flexion of lumbar spine at) 20° involving such a captured motion. Such feedback may be in the form of video, audio, and/or haptic alerts. A further discussion regarding these and other features is provided below.

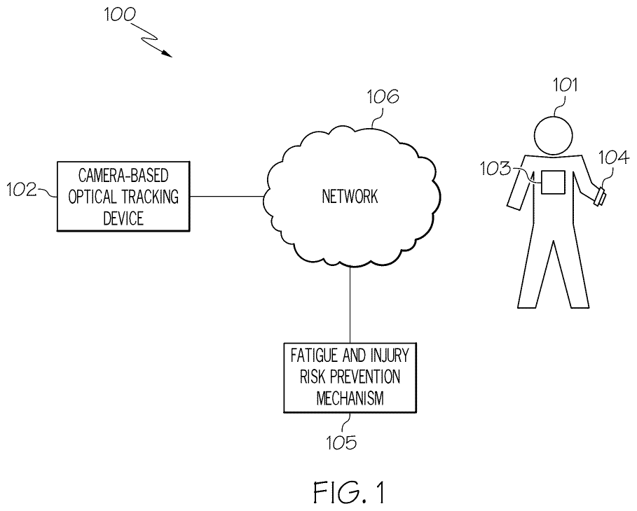

Referring now to the Figures in detail, FIG. 1 illustrates an embodiment of the present disclosure of a system 100 for predicting fatigue and injury risk of a user 101 (e.g., worker performing material handling operations). System 100 includes a camera-based optical tracking device 102 configured to monitor and capture motions of user 101. Such motions include lifting, carrying, and manipulating objects, such as redistributing, moving, and retrieving goods within the warehouse. Optical tracking device 102, as used herein, uses multiple cameras to triangulate the 3D location and movement of objects, such as user 101, who may be present in a warehouse environment. In one embodiment, such cameras are RGB cameras.

In one embodiment, camera-based optical tracking device 102 monitors and captures the motions of user 101 by determining in real-time the position of user 101 by tracking the positions of either active or passive infrared markers attached to user 101. Examples of camera-based optical tracking device 102 include, but are not limited to, spryTrack®, optiTrack®, Microsoft® Kinect®, etc.

In one embodiment, camera-based optical tracking device 102 utilizes motion tracking software to monitor and track the motions of user 101. Examples of such motion tracking software include, but are not limited to, Boris FX® Mocha Pro, Blender, Da Vinci Resolve®, Adobe® After Effects®, Filmora®, etc.

The motions of user 101 may be monitored and captured using other types of technology, such as an electromyography (EMG) sensor 103 worn by user 101. Electromyography sensor 103, as used herein, refers to a sensor that measures small electromyographic signals generated by the movement of muscles of user 101. Based on such measurements, fatigue and injury risk prevention mechanism 105, discussed further below, determines the movements of user 101. In one embodiment, a machine learning model is trained to predict the user's motion intention based on electromyographic signals. Based on such a trained model, fatigue and injury risk prevention mechanism 105 detects the user's motion intention based on the electromyographic signals from electromyography sensor 103. Examples of electromyography sensor 103 include, but are not limited to, Trigno® EMG (electromyography) sensors, Shimmer3 EMG unit, etc.

The motions of user 101 may be monitored and captured using inertia measurement unit (IMU) 104, which may be worn by user 101, such as on the wrist of user 101. Inertia measurement unit 104, as used herein, is an electronic device that measures and reports acceleration, orientation, angular rates, and other gravitational forces. Examples of inertia measurement unit 104 include, but are not limited to, Shimmer3 Consensys IMU, Bosch® BMI270, etc.

In one embodiment, based on measurements from inertia measurement unit 104, fatigue and injury risk prevention mechanism 105, discussed further below, determines the movements of user 101. In one embodiment, a machine learning model is trained to predict the user's motion intention based on such measurements. Based on such a trained model, fatigue and injury risk prevention mechanism 105 detects the user's motion intention based on measurements from inertia measurement unit 104.

Furthermore, as illustrated in FIG. 1, measurements from camera-based optical tracking device 102, electromyography sensor 103, and inertia measurement unit 104 are provided to fatigue and injury risk prevention mechanism 105 via network 106. Network 106 may be, for example, a local area network, a wide area network, a wireless wide area network, a circuit-switched telephone network, a Global System for Mobile communications (GSM) network, a Wireless Application Protocol (WAP) network, a WiFi network, an IEEE 802.11 standards network, various combinations thereof, etc. Other networks, whose descriptions are omitted here for brevity, may also be used in conjunction with system 100 of FIG. 1 without departing from the scope of the present disclosure.

Referring again to FIG. 1, fatigue and injury risk prevention mechanism 105 is configured to predict fatigue and injury risk to user 101, such as a worker performing material handling operations, using a trained machine learning model to predict fatigue and injury risk to users based on the captured motions from camera-based optical tracking device 102, electromyography sensor 103 and/or inertia measurement unit 104. In one embodiment, such a prediction is made by the trained machine learning model by comparing the captured motions to predefined thresholds of range of motion constraints based on biomechanical parameters of the user from a digital twin model of the user. Range of motion constraints, as used herein, refer to the limitations or restrictions placed on the movement of a joint or body part, defining the maximum extent to which it can move in a specific direction, in order to prevent fatigue or injury risk. Biomechanical parameters, as used herein, refer to characteristics relating to the function and motion of the mechanical aspects of users. A digital twin model, as used herein, refers to a digital model of the user that serves as the effectively indistinguishable digital counterpart of the user for practical purposes, such as simulation, testing, and monitoring.

In one embodiment, fatigue and injury risk prevention mechanism 105 builds a digital twin model of user 101 using various software tools, such as Ansys® Twin Builder, HelixCore, Simio® Simulation, vHive, Veerum, etc. based on real-world data about user 101, which may be acquired from camera-based optical tracking device 102, electromyography sensor 103 and/or inertia measurement unit 104. Such data may include 3D data on the user's body movements during the user's work tasks as well as the associated motion constraints for such body movements. In another embodiment, such data used to create the digital twin model of user 101 is inputted by an expert and/or by user 101.

In one embodiment, the biomechanical parameters of user 101 are acquired from user 101 and/or an expert. Examples of biomechanical parameters include, but are not limited to, height, limb length, and torque limits.

In one embodiment, predefined thresholds for range of motion constraints, which may be established by experts based on the biomechanical parameters of user 101 from a digital twin model of user 101, are used to determine if user 101 is subject to a risk of fatigue and/or injury risk using the trained model.

In one embodiment, fatigue and injury risk prevention mechanism 105 provides feedback based on the trained model's prediction of fatigue and injury risk of user 101. Such feedback may be in the form of video, audio, and/or haptic alerts. For example, feedback concerning user 101 being subject to the risk of fatigue and/or injury risk may be provided in response to a captured motion (right lateral flexion of lumbar spine at) 25° exceeding a predefined threshold of a range of motion constraint (right lateral flexion of lumbar spine at) 20° involving such a captured motion.

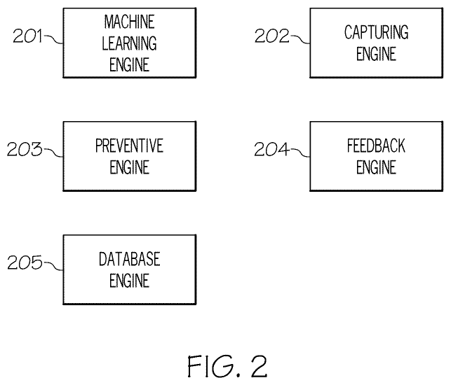

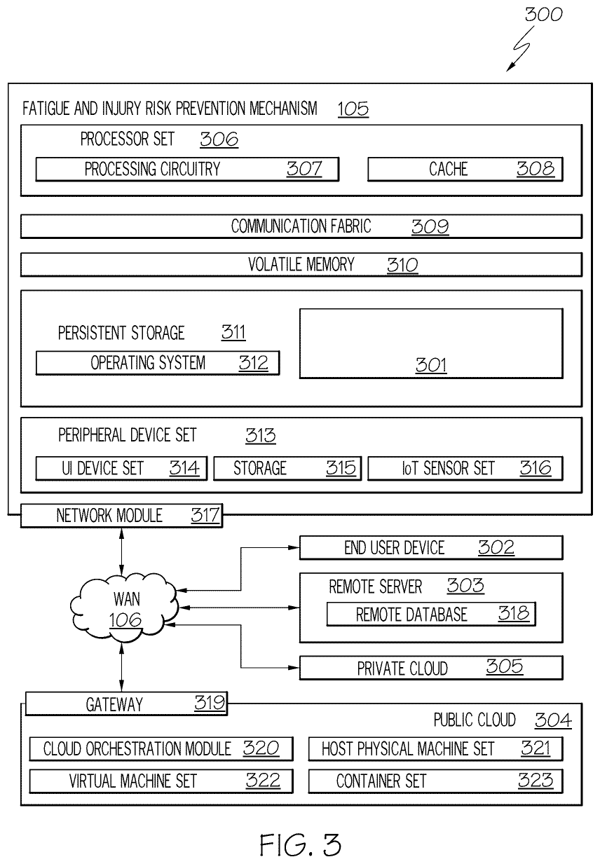

A description of the software components of fatigue and injury risk prevention mechanism 105 used for predicting fatigue and injury risk to user 101, such as a worker performing material handling operations, is provided below in connection with FIG. 2. A description of the hardware configuration of fatigue and injury risk prevention mechanism 105 is provided further below in connection with FIG. 3.

System 100 is not to be limited in scope to any one particular network architecture. System 100 may include any number of users 101, camera-based optical tracking devices 102, electromyography (EMG) sensors 103, inertia measurement units 104, fatigue and injury risk prevention mechanisms 105, and networks 106.

A discussion regarding the software components used by fatigue and injury risk prevention mechanism 105 for predicting fatigue and injury risk to users, such as a worker performing material handling operations, is provided below in connection with FIG. 2.

FIG. 2 is a diagram of the software components used by fatigue and injury risk prevention mechanism 105 for predicting fatigue and injury risk to users, such as a worker performing material handling operations, in accordance with an embodiment of the present disclosure.

Referring to FIG. 2, in conjunction with FIG. 1, fatigue and injury risk prevention mechanism 105 includes machine learning engine 201 configured to build and train a machine learning model to predict fatigue and injury risk for users, such as workers performing material handling operations, in real-time. That is, machine learning engine 201 uses a machine learning algorithm (e.g., supervised learning) to build and train a machine learning model for predicting fatigue and injury risk to workers, such as a worker performing material handling operations, using training data consisting of motions, range of motion constraints, biomechanical parameters, and predefined thresholds of range of motion constraints. Range of motion constraints, as used herein, refer to the limitations or restrictions placed on the movement of a joint or body part, defining the maximum extent to which it can move in a specific direction, in order to prevent fatigue or injury risk. Biomechanical parameters, as used herein, refer to characteristics relating to the function and motion of the mechanical aspects of users. In one embodiment, the biomechanical parameters of user 101 are acquired from user 101 and/or an expert. Examples of biomechanical parameters include, but are not limited to, height, limb length, and torque limits. In one embodiment, such training data is populated by an expert.

In one embodiment, the training data discussed above is used by the machine learning algorithm to predict fatigue and injury risk for users, such as workers performing material handling operations, in real-time. The algorithm iteratively predicts fatigue and injury risk for users, such as workers performing material handling operations, using the training data until the predictions achieve the desired accuracy as determined by an expert. Examples of such learning algorithms include nearest neighbor, Naïve Bayes, decision trees, linear regression, support vector machines, deep learning, and neural networks. In one embodiment, the machine learning algorithm is a long-short-term memory recurrent neural network.

Upon training the machine learning model to predict fatigue and injury risk for users, such a model is used to predict fatigue and injury risk for user 101 based on comparing the captured motions to predefined thresholds for range of motion constraints, which may be established by experts based on the biomechanical parameters of user 101 from a digital twin model of user 101.

Furthermore, fatigue and injury risk prevention mechanism 105 includes a capturing engine 202 configured to capture motions performed by user 101. Such motions include lifting, carrying, and manipulating objects, such as redistributing, moving, and retrieving goods within the warehouse.

In one embodiment, capturing engine 202 captures motions performed by user 101 by receiving inputs from camera-based optical tracking device 102, electromyography (EMG) sensor 103, and inertia measurement unit 104.

Camera-based optical tracking device 102, as used herein, uses multiple cameras to triangulate the 3D location and movement of objects, such as user 101, who may be present in a warehouse environment. In one embodiment, such cameras are RGB cameras.

In one embodiment, camera-based optical tracking device 102 monitors and captures the motions of user 101 by determining in real-time the position of user 101 by tracking the positions of either active or passive infrared markers attached to user 101. Examples of optical tracking device 102 include, but are not limited to, spryTrack®, optiTrack®, Microsoft® Kinect®, etc.

In one embodiment, camera-based optical tracking device 102 utilizes motion tracking software to monitor and track the motions of user 101. Examples of such motion tracking software include, but are not limited to, Boris FX® Mocha Pro, Blender, Da Vinci Resolve®, Adobe® After Effects®, Filmora®, etc.

Electromyography sensor 103, as used herein, refers to a sensor that measures small electromyographic signals generated by the movement of muscles of user 101. Based on such measurements, fatigue and injury risk prevention mechanism 105, discussed further below, determines the movements of user 101. In one embodiment, machine learning engine 201 is configured to build and train a machine learning model to predict the user's motion intention based on electromyographic signals. That is, machine learning engine 201 uses a machine learning algorithm (e.g., supervised learning) to build and train a machine learning model for predicting the user's motion intention using training data consisting of electromyographic signals. In one embodiment, such training data is populated by an expert.

In one embodiment, the training data is used by the machine learning algorithm to predict the user's motion intention based on electromyographic signals. The algorithm iteratively predicts the user's motion intention based on electromyographic signals until the predictions achieve the desired accuracy as determined by an expert. Examples of such learning algorithms include nearest neighbor, Naïve Bayes, decision trees, linear regression, support vector machines, deep learning, and neural networks.

Based on such a trained model, capturing engine 202 detects the user's motion intention based on the electromyographic signals from electromyography sensor 103. Examples of electromyography sensor 103 include, but are not limited to, Trigno® EMG (electromyography) sensors, Shimmer3 EMG unit, etc.

Furthermore, inertia measurement unit 104, as used herein, is an electronic device that measures and reports acceleration, orientation, angular rates, and other gravitational forces. Examples of inertia measurement units 104 include, but are not limited to, Shimmer3 Consensys IMU, Bosch® BMI270, etc.

In one embodiment, based on measurements from inertia measurement unit 104, capturing engine 202 determines the movements of user 101.

In one embodiment, machine learning engine 201 is configured to build and train a machine learning model to predict the user's motion intention based on measurements from inertia measurement unit 104. That is, machine learning engine 201 uses a machine learning algorithm (e.g., supervised learning) to build and train a machine learning model for predicting the user's motion intention using training data consisting of measurements from inertia measurement unit 104. In one embodiment, such training data is populated by an expert.

In one embodiment, the training data is used by the machine learning algorithm to predict the user's motion intention based on measurements from inertia measurement unit 104. The algorithm iteratively predicts the user's motion intention based on measurements from inertia measurement unit 104 until the predictions achieve the desired accuracy as determined by an expert. Examples of such learning algorithms include nearest neighbor, Naïve Bayes, decision trees, linear regression, support vector machines, deep learning, and neural networks.

Based on such a trained model, capturing engine 202 detects the user's motion intention based on measurements from inertia measurement unit 104.

Fatigue and injury risk prevention mechanism 105 further includes preventive engine 203 configured to predict fatigue and injury risk to user 101, such as a worker performing material handling operations, using the trained model for predicting fatigue and injury risk to users based on the captured motions of user 101 from camera-based optical tracking device 102, electromyography sensor 103 and/or inertia measurement unit 104. In one embodiment, preventive engine 203 uses the trained model to predict fatigue and injury risk to users by comparing the captured motions of user 101 to predefined thresholds of range of motion constraints based on the biomechanical parameters of user 101 from a digital twin model of user 101. Range of motion constraints, as used herein, refer to the limitations or restrictions placed on the movement of a joint or body part, defining the maximum extent to which it can move in a specific direction, in order to prevent fatigue or injury risk. Biomechanical parameters, as used herein, refer to characteristics relating to the function and motion of the mechanical aspects of users. A digital twin model, as used herein, refers to a digital model of the user that serves as the effectively indistinguishable digital counterpart of the user for practical purposes, such as simulation, testing, and monitoring.

In one embodiment, preventive engine 203 builds a digital twin model of user 101 using various software tools, such as Ansys® Twin Builder, HelixCore, Simio® Simulation, vHive, Veerum, etc. based on real-world data about user 101, which may be acquired from camera-based optical tracking device 102, electromyography sensor 103 and/or inertia measurement unit 104. Such data may include 3D data on the user's body movements during the user's work tasks as well as the associated motion constraints for such body movements. In another embodiment, such data used to create the digital twin model of user 101 is inputted by an expert and/or by user 101.

In one embodiment, the biomechanical parameters of user 101 are acquired from user 101 and/or an expert. Examples of biomechanical parameters include, but are not limited to, height, limb length, and torque limits.

In one embodiment, predefined thresholds for range of motion constraints, which may be established by experts based on the biomechanical parameters of user 101 from a digital twin model of user 101, are used to determine if user 101 is subject to a risk of fatigue and/or injury risk using the trained model.

In one embodiment, predefined thresholds for range of motion constraints are based on the baseline fatigue levels and the baseline motions of user 101.

In one embodiment, preventive engine 203 saves the motions of user 101 captured by capturing engine 202. In one embodiment, such captured motions are saved in a storage device of fatigue and injury risk preventive mechanism 105.

In one embodiment, preventive engine 203 is configured to identify trends from the saved captured motions using various software tools, including, but are not limited to, R, SPSS®, Stata® etc.

In one embodiment, preventive engine 203 modifies the baseline fatigue levels and the baseline motions of user 101 based on such trends. For example, if the trends indicate a peak trunk range of motion with 32° axial rotation and a 59° flexion-extension, then such measurements may be used to determine the predefined thresholds for the constraints of a trunk motion for an axial rotation and flexion-extension.

Furthermore, fatigue and injury risk preventive mechanism 105 includes feedback engine 204 for providing feedback based on the trained model's prediction of fatigue and injury risk of user 101. Such feedback may be in the form of video, audio, and/or haptic alerts. For example, feedback concerning user 101 being subject to the risk of fatigue and/or injury risk may be provided in response to a captured motion (right lateral flexion of lumbar spine at) 25° exceeding a predefined threshold of a range of motion constraint (right lateral flexion of lumbar spine at) 20° involving such a captured motion.

Additionally, fatigue and injury risk preventive mechanism 105 includes database engine 205 which is used by a database management system to create, read, update, and delete data from a database.

A further description of these and other features is provided below in connection with the discussion of the method for predicting fatigue and injury risk to users, such as a worker performing material handling operations.

Prior to the discussion of the method for predicting fatigue and injury risk to users, a description of the hardware configuration of fatigue and injury risk prevention mechanism 105 (FIG. 1) is provided below in connection with FIG. 3.

Referring now to FIG. 3, in conjunction with FIG. 1, FIG. 3 illustrates an embodiment of the present disclosure of the hardware configuration of fatigue and injury risk prevention mechanism 105 which is representative of a hardware environment for practicing the present disclosure.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 300 contains an example of an environment for the execution of at least some of the computer code 301 involved in performing the inventive methods, such as predicting fatigue and injury risk for users in real-time. In addition to block 301, computing environment 300 includes, for example, fatigue and injury risk prevention mechanism 105, network 106, such as a wide area network (WAN), end user device (EUD) 302, remote server 303, public cloud 304, and private cloud 305. In this embodiment, fatigue and injury risk prevention mechanism 105 includes processor set 306 (including processing circuitry 307 and cache 308), communication fabric 309, volatile memory 310, persistent storage 311 (including operating system 312 and block 301, as identified above), peripheral device set 313 (including user interface (UI) device set 314, storage 315, and Internet of Things (IoT) sensor set 316), and network module 317. Remote server 303 includes remote database 318. Public cloud 304 includes gateway 319, cloud orchestration module 320, host physical machine set 321, virtual machine set 322, and container set 323.

Fatigue and injury risk prevention mechanism 105 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 318. As is well understood in the art of computer technology, and depending upon the technology, performance of a computer-implemented method may be distributed among multiple computers and/or between multiple locations. On the other hand, in this presentation of computing environment 300, detailed discussion is focused on a single computer, specifically fatigue and injury risk prevention mechanism 105, to keep the presentation as simple as possible. Fatigue and injury risk prevention mechanism 105 may be located in a cloud, even though it is not shown in a cloud in FIG. 3. On the other hand, fatigue and injury risk prevention mechanism 105 is not required to be in a cloud except to any extent as may be affirmatively indicated.

Processor set 306 includes one, or more, computer processors of any type now known or to be developed in the future. Processing circuitry 307 may be distributed over multiple packages, for example, multiple, coordinated integrated circuit chips. Processing circuitry 307 may implement multiple processor threads and/or multiple processor cores. Cache 308 is memory that is located in the processor chip package(s) and is typically used for data or code that should be available for rapid access by the threads or cores running on processor set 306. Cache memories are typically organized into multiple levels depending upon relative proximity to the processing circuitry. Alternatively, some, or all, of the cache for the processor set may be located “off chip.” In some computing environments, processor set 306 may be designed for working with qubits and performing quantum computing.

Computer readable program instructions are typically loaded onto fatigue and injury risk prevention mechanism 105 to cause a series of operational steps to be performed by processor set 306 of fatigue and injury risk prevention mechanism 105 and thereby effect a computer-implemented method, such that the instructions thus executed will instantiate the methods specified in flowcharts and/or narrative descriptions of computer-implemented methods included in this document (collectively referred to as “the disclosed methods”). These computer readable program instructions are stored in various types of computer readable storage media, such as cache 308 and the other storage media discussed below. The program instructions, and associated data, are accessed by processor set 306 to control and direct performance of the disclosed methods. In computing environment 300, at least some of the instructions for performing the disclosed methods may be stored in block 301 in persistent storage 311. Communication fabric 309 is the signal conduction paths that allow the various components of fatigue and injury risk prevention mechanism 105 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 310 is any type of volatile memory now known or to be developed in the future. Examples include dynamic type random access memory (RAM) or static type RAM. Typically, the volatile memory is characterized by random access, but this is not required unless affirmatively indicated. In fatigue and injury risk prevention mechanism 105, the volatile memory 310 is located in a single package and is internal to fatigue and injury risk prevention mechanism 105, but, alternatively or additionally, the volatile memory may be distributed over multiple packages and/or located externally with respect to fatigue and injury risk prevention mechanism 105.

Persistent Storage 311 is any form of non-volatile storage for computers that is now known or to be developed in the future. The non-volatility of this storage means that the stored data is maintained regardless of whether power is being supplied to fatigue and injury risk prevention mechanism 105 and/or directly to persistent storage 311. Persistent storage 311 may be a read only memory (ROM), but typically at least a portion of the persistent storage allows writing of data, deletion of data and re-writing of data. Some familiar forms of persistent storage include magnetic disks and solid state storage devices. Operating system 312 may take several forms, such as various known proprietary operating systems or open source Portable Operating System Interface type operating systems that employ a kernel. The code included in block 301 typically includes at least some of the computer code involved in performing the disclosed methods.

Peripheral device set 313 includes the set of peripheral devices of fatigue and injury risk prevention mechanism 105. Data communication connections between the peripheral devices and the other components of fatigue and injury risk prevention mechanism 105 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 314 may include components such as a display screen, speaker, microphone, wearable devices (such as goggles and smart watches), keyboard, mouse, printer, touchpad, game controllers, and haptic devices. Storage 315 is external storage, such as an external hard drive, or insertable storage, such as an SD card. Storage 315 may be persistent and/or volatile. In some embodiments, storage 315 may take the form of a quantum computing storage device for storing data in the form of qubits. In embodiments where fatigue and injury risk prevention mechanism 105 is required to have a large amount of storage (for example, where fatigue and injury risk prevention mechanism 105 locally stores and manages a large database) then this storage may be provided by peripheral storage devices designed for storing very large amounts of data, such as a storage area network (SAN) that is shared by multiple, geographically distributed computers. IoT sensor set 316 is made up of sensors that can be used in Internet of Things applications. For example, one sensor may be a thermometer and another sensor may be a motion detector.

Network module 317 is the collection of computer software, hardware, and firmware that allows fatigue and injury risk prevention mechanism 105 to communicate with other computers through WAN 106. Network module 317 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 317 are performed on the same physical hardware device. In other embodiments (for example, embodiments that utilize software-defined networking (SDN)), the control functions and the forwarding functions of network module 317 are performed on physically separate devices, such that the control functions manage several different network hardware devices. Computer readable program instructions for performing the disclosed methods can typically be downloaded to fatigue and injury risk prevention mechanism 105 from an external computer or external storage device through a network adapter card or network interface included in network module 317.

WAN 106 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 302 is any computer system that is used and controlled by an end user (for example, a customer of an enterprise that operates fatigue and injury risk prevention mechanism 105), and may take any of the forms discussed above in connection with fatigue and injury risk prevention mechanism 105. EUD 302 typically receives helpful and useful data from the operations of fatigue and injury risk prevention mechanism 105. For example, in a hypothetical case where fatigue and injury risk prevention mechanism 105 is designed to provide a recommendation to an end user, this recommendation would typically be communicated from network module 317 of fatigue and injury risk prevention mechanism 105 through WAN 106 to EUD 302. In this way, EUD 302 can display, or otherwise present, the recommendation to an end user. In some embodiments, EUD 302 may be a client device, such as thin client, heavy client, mainframe computer, desktop computer and so on.

Remote server 303 is any computer system that serves at least some data and/or functionality to fatigue and injury risk prevention mechanism 105. Remote server 303 may be controlled and used by the same entity that operates fatigue and injury risk prevention mechanism 105. Remote server 303 represents the machine(s) that collect and store helpful and useful data for use by other computers, such as fatigue and injury risk prevention mechanism 105. For example, in a hypothetical case where fatigue and injury risk prevention mechanism 105 is designed and programmed to provide a recommendation based on historical data, then this historical data may be provided to fatigue and injury risk prevention mechanism 105 from remote database 318 of remote server 303.

Public cloud 304 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 304 is performed by the computer hardware and/or software of cloud orchestration module 320. The computing resources provided by public cloud 304 are typically implemented by virtual computing environments that run on various computers making up the computers of host physical machine set 321, which is the universe of physical computers in and/or available to public cloud 304. The virtual computing environments (VCEs) typically take the form of virtual machines from virtual machine set 322 and/or containers from container set 323. It is understood that these VCEs may be stored as images and may be transferred among and between the various physical machine hosts, either as images or after instantiation of the VCE. Cloud orchestration module 320 manages the transfer and storage of images, deploys new instantiations of VCEs and manages active instantiations of VCE deployments. Gateway 319 is the collection of computer software, hardware, and firmware that allows public cloud 304 to communicate through WAN 106.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 305 is similar to public cloud 304, except that the computing resources are only available for use by a single enterprise. While private cloud 305 is depicted as being in communication with WAN 106 in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 304 and private cloud 305 are both part of a larger hybrid cloud.

Block 301 further includes the software components discussed above in connection with FIG. 2 to predict fatigue and injury risk for users in real-time. In one embodiment, such components may be implemented in hardware. The functions discussed above performed by such components are not generic computer functions. As a result, fatigue and injury risk prevention mechanism 105 is a particular machine that is the result of implementing specific, non-generic computer functions.

In one embodiment, the functionality of such software components of fatigue and injury risk prevention mechanism 105, including the functionality for predicting fatigue and injury risk for users in real-time, may be embodied in an application specific integrated circuit.

As stated above, workers within industrial sectors, such as manufacturing, warehousing, shipping and logistics, construction, and mining, face numerous hazards during daily operations. Globally, tens of millions of workers in manually intensive jobs experience musculoskeletal disorders and injuries from occupational hazards, which causes billions in healthcare costs annually. Specifically, in manufacturing, ergonomic hazards for assembly workers who perform repetitive motions over long hours are of particular concern and can lead to overuse injuries. In warehousing and shipping, order fulfillment roles involve substantial manual material handling, lifting, bending, and twisting motions that strain the body and cause frequent sprains and falls. In construction and mining, workers are exposed to especially significant safety risks while working with heavy machinery and in dangerous environments. There is a substantial need and incentive for companies in these industries to invest in safety monitoring tools to protect their workers. As a result, there have been attempts to improve workplace safety and ergonomics by detecting potentially harmful motions or excessive fatigue. For example, methods, such as standard questionnaires after completing a job, have been used in the past to analyze fatigue in construction workers. Unfortunately, relying on such questionnaires only addresses fatigue and injuries after such occurrences. Various ergonomic assessment tools, such as Rapid Upper Limb Assessment (RULA) and the job strain index method, are commonly practiced in industries to identify repetitive movements. In order to identify strained postures, observational tools, such as Rapid Entire Body Assessment (REBA) and the Ovako Working Assessment System (OWAS), provide feedback based on an experienced user's scoring system. The National Institute for Occupational Safety and Health (NIOSH) lifting equation, Snook tables, and Liberty Mutual tables provide information on safe load capacity. The commonly used Borg scale assess fatigue by subjective worker feedback. Such methods require post-experiment evaluation resulting in an inability to provide real-time feedback to the operator. Furthermore, another limitation of currently used methods is not including the object's weight, a significant component of manual material handling (MMH) tasks, as a decision variable. In recent times, virtual human factor (VHF) tools such as virtual reality, digital human models, and discrete event simulation allow the user to perform an ergonomic assessment to systems not yet constructed. However, such tools posses the limitation of being used only in the design stage of the process. Furthermore, such tools may be limited to light assembly work and only considering loads at the shoulder joint. Unfortunately, such current tools are deficient in predicting fatigue and injury risk for users, such as workers performing material handling operations, in real-time.

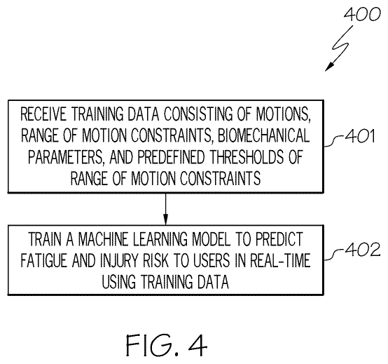

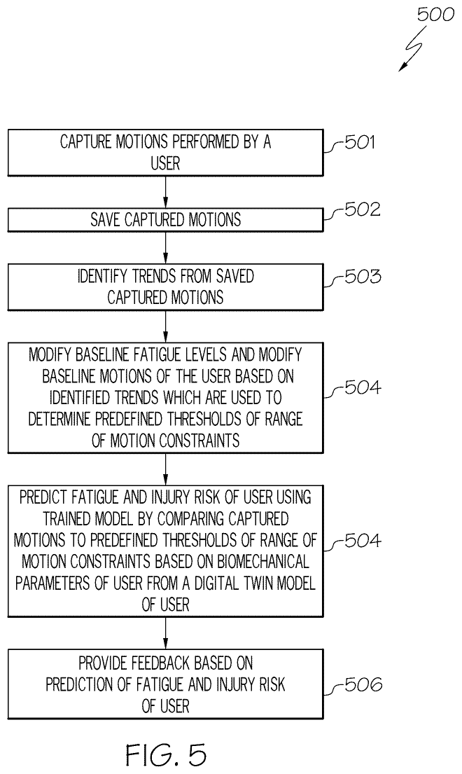

The embodiments of the present disclosure provide a means for predicting fatigue and injury risk for users, such as workers performing material handling operations, in real-time as discussed below in connection with FIGS. 4-5. FIG. 4 is a flowchart of a method for training a machine learning model to predict fatigue and injury risk for users in real-time. FIG. 5 is a flowchart of a method for predicting fatigue and injury risk for users in real-time.

As stated above, FIG. 4 is a flowchart of a method 400 for training a machine learning model to predict fatigue and injury risk for users in real-time in accordance with an embodiment of the present disclosure.

Referring to FIG. 4, in conjunction with FIGS. 1-3, in step 401, machine learning engine 201 of fatigue and injury risk prevention mechanism 105 receives training data consisting of motions, range of motion constraints, biomechanical parameters, and predefined thresholds of range of motion constraints. Range of motion constraints, as used herein, refer to the limitations or restrictions placed on the movement of a joint or body part, defining the maximum extent to which it can move in a specific direction, in order to prevent fatigue or injury risk. Biomechanical parameters, as used herein, refer to characteristics relating to the function and motion of the mechanical aspects of users. In one embodiment, the biomechanical parameters of user 101 are acquired from user 101 and/or an expert. Examples of biomechanical parameters include, but are not limited to, height, limb length, and torque limits.

In step 402, machine learning engine 201 of fatigue and injury risk prevention mechanism 105 trains a machine learning model to predict fatigue and injury risk for users, such as workers performing material handling operations, in real-time using the training data.

As discussed above, machine learning engine 201 builds and trains a machine learning model to predict fatigue and injury risk for users, such as workers performing material handling operations, in real-time. That is, machine learning engine 201 uses a machine learning algorithm (e.g., supervised learning) to build and train a machine learning model for predicting fatigue and injury risk to workers, such as a worker performing material handling operations, using training data consisting of motions, range of motion constraints, biomechanical parameters, and predefined thresholds of range of motion constraints. Range of motion constraints, as used herein, refer to the limitations or restrictions placed on the movement of a joint or body part, defining the maximum extent to which it can move in a specific direction, in order to prevent fatigue or injury risk. Biomechanical parameters, as used herein, refer to characteristics relating to the function and motion of the mechanical aspects of users. In one embodiment, the biomechanical parameters of user 101 are acquired from user 101 and/or an expert. Examples of biomechanical parameters include, but are not limited to, height, limb length, and torque limits. In one embodiment, such training data is populated by an expert.

In one embodiment, the training data discussed above is used by the machine learning algorithm to predict fatigue and injury risk for users, such as workers performing material handling operations, in real-time. The algorithm iteratively predicts fatigue and injury risk for users, such as workers performing material handling operations, using the training data until the predictions achieve the desired accuracy as determined by an expert. Examples of such learning algorithms include nearest neighbor, Naïve Bayes, decision trees, linear regression, support vector machines, deep learning, and neural networks. In one embodiment, the machine learning algorithm is a long-short-term memory recurrent neural network.

Upon training the machine learning model to predict fatigue and injury risk for users, such a model is used to predict fatigue and injury risk for user 101 based on comparing the captured motions to predefined thresholds of range of motion constraints, which may be established by experts based on biomechanical parameters of user 101 from a digital twin model of user 101 as discussed below in connection with FIG. 5.

FIG. 5 is a flowchart of a method 500 for predicting fatigue and injury risk for users in real-time in accordance with an embodiment of the present disclosure.

Referring to FIG. 5, in conjunction with FIGS. 1-4, in step 501, capturing engine 202 of fatigue and injury risk prevention mechanism 105 captures motions performed by user 101.

As stated above, capturing engine 202 captures motions performed by user 101, where such motions include lifting, carrying, and manipulating objects, such as redistributing, moving, and retrieving goods within the warehouse.

In one embodiment, capturing engine 202 captures motions performed by user 101 by receiving inputs from camera-based optical tracking device 102, electromyography (EMG) sensor 103, and inertia measurement unit 104.

Camera-based optical tracking device 102, as used herein, uses multiple cameras to triangulate the 3D location and movement of objects, such as user 101, who may be present in a warehouse environment. In one embodiment, such cameras are RGB cameras.

In one embodiment, camera-based optical tracking device 102 monitors and captures the motions of user 101 by determining in real-time the position of user 101 by tracking the positions of either active or passive infrared markers attached to user 101. Examples of camera-based optical tracking device 102 include, but are not limited to, spryTrack®, optiTrack®, Microsoft® Kinect®, etc.

In one embodiment, camera-based optical tracking device 102 utilizes motion tracking software to monitor and track the motions of user 101. Examples of such motion tracking software include, but are not limited to, Boris FX® Mocha Pro, Blender, DaVinci Resolve®, Adobe® After Effects®, Filmora®, etc.

Electromyography sensor 103, as used herein, refers to a sensor that measures small electromyographic signals generated by the movement of muscles of user 101. Based on such measurements, fatigue and injury risk prevention mechanism 105, discussed further below, determines the movements of user 101. In one embodiment, machine learning engine 201 is configured to build and train a machine learning model to predict the user's motion intention based on electromyographic signals. That is, machine learning engine 201 uses a machine learning algorithm (e.g., supervised learning) to build and train a machine learning model for predicting the user's motion intention using training data consisting of electromyographic signals. In one embodiment, such training data is populated by an expert.

In one embodiment, the training data is used by the machine learning algorithm to predict the user's motion intention based on electromyographic signals. The algorithm iteratively predicts the user's motion intention based on electromyographic signals until the predictions achieve the desired accuracy as determined by an expert. Examples of such learning algorithms include nearest neighbor, Naïve Bayes, decision trees, linear regression, support vector machines, deep learning, and neural networks.

Based on such a trained model, capturing engine 202 detects the user's motion intention based on the electromyographic signals from electromyography sensor 103. Examples of electromyography sensor 103 include, but are not limited to, Trigno® EMG (electromyography) sensors, Shimmer3 EMG unit, etc.

Furthermore, inertia measurement unit 104, as used herein, is an electronic device that measures and reports acceleration, orientation, angular rates, and other gravitational forces. Examples of inertia measurement units 104 include, but are not limited to, Shimmer3 Consensys IMU, Bosch® BMI270, etc.

In one embodiment, based on measurements from inertia measurement unit 104, capturing engine 202 determines the movements of user 101.

In one embodiment, machine learning engine 201 is configured to build and train a machine learning model to predict the user's motion intention based on measurements from inertia measurement unit 104. That is, machine learning engine 201 uses a machine learning algorithm (e.g., supervised learning) to build and train a machine learning model for predicting the user's motion intention using training data consisting of measurements from inertia measurement unit 104. In one embodiment, such training data is populated by an expert.

In one embodiment, the training data is used by the machine learning algorithm to predict the user's motion intention based on measurements from inertia measurement unit 104. The algorithm iteratively predicts the user's motion intention based on measurements from inertia measurement unit 104 until the predictions achieve the desired accuracy as determined by an expert. Examples of such learning algorithms include nearest neighbor, Naïve Bayes, decision trees, linear regression, support vector machines, deep learning, and neural networks.

Based on such a trained model, capturing engine 202 detects the user's motion intention based on measurements from inertia measurement unit 104.

In step 502, capturing engine 202 of fatigue and injury risk prevention mechanism 105 saves the motions captured by capturing engine 202. In one embodiment, such captured motions are saved in a storage device (e.g., storage device 311, 315) of fatigue and injury risk preventive mechanism 105.

In step 503, preventive engine 203 of fatigue and injury risk prevention mechanism 105 identifies trends from the saved captured motions.

In one embodiment, preventive engine 203 analyzes the data from the captured motions (e.g., captured motions saved in the storage device of fatigue and injury risk preventive mechanism 105) using statistical methods to identify patterns and recurring behaviors, such as the frequency, speed, and direction of movements, which can reveal trends related to user interface with a product or interface, potential usability issues, etc.

In one embodiment, preventive engine 203 performs feature extraction from the data. In one embodiment, preventive engine 203 identifies relevant features from the motion data, such as joint angles, velocity, acceleration, and movement patterns.

In one embodiment, preventive engine 203 performs a statistical analysis on the data from the captured motions. In one embodiment, such a statistical analysis includes time series analysis. For example, preventive engine 203 analyzes how user motions change over time to identify trends and patterns.

In one embodiment, such a statistical analysis includes clustering analysis. For example, preventive engine 203 groups similar motion patterns together to identify distinct user behaviors.

In one embodiment, such a statistical analysis includes regression analysis. For example, preventive engine 203 investigates relationships between user motions and other factors, such as task complexity or user experience.

Examples of trends identified by preventive engine 203 include frequent reaching motions (e.g., users reaching for a specific button indicating potential layout issues), hesitation in movements (e.g., frequent pauses during a task could suggest confusion or difficulty with the interaction), and repetitive strain patterns, which can lead to user fatigue or discomfort.

Furthermore, preventive engine 203 identifies such trends from the saved captured motions using various software tools, including, but are not limited to, R, SPSS®, Stata®, etc.

In step 504, preventive engine 203 of fatigue and injury risk prevention mechanism 105 modifies the baseline fatigue levels and modifies the baseline motions of user 101 based on such trends, where such baseline fatigue levels and baseline motions of user 101 are used to determine predefined thresholds of the range of motion constraints. Baseline fatigue levels, as used herein, refer to the user's typical or normal level of tiredness or exhaustion. That is, the baseline fatigue level of the user (e.g., user 101) refers to the level of fatigue the user experiences on a regular basis without any significant external factors influencing it. The baseline motions of the user, as used herein, refer to the natural, everyday movements a person performs without any intentional effort to maximize or restrict their range of motion.

In one embodiment, preventive engine 203 modifies the baseline fatigue levels based on the identified trends by actively changing or adjusting the level of tiredness or weariness a person typically experiences at their most rested state. Physical fatigue significantly limits the range of movement of the user, such as at a joint. For example, a person cannot move their body part as far as they normally would due to muscle tiredness leading to decreased flexibility and potential for injury. That is, when muscles are fatigued, they cannot contract as effectively thereby restricting joint mobility. For instance, when muscles are fatigued, they produce less force and can shorten less which directly impacts the range of motion at a joint. As a result, a predefined threshold of a range of motion constraint is directly impacted by the baseline fatigue level of the user.

In one embodiment, preventive engine 203 modifies the baseline motions of the user based on the identified trends which indicate the user's typical movement patterns (their “baseline motions”), which establish limits on how far their joints can move thereby establishing the predefined thresholds of the range of motivation constraints. That is, based on such baseline motions, parameters are established for the user's range of motion to prevent unnatural or potentially harmful movements based on their individual capabilities.

An example of modifying the baseline fatigue levels and baseline motions of the user based on the identified trends, which are used to determine predefined thresholds of the range of motion constraints is the following. If the trends indicate a peak trunk range of motion with 32° axial rotation and 59° flexion-extension, then such measurements may be used to determine the predefined thresholds for the constraints of a trunk motion for an axial rotation and flexion-extension.

Furthermore, preventive engine 203 modifies the baseline fatigue levels and the baseline motions of the user based on the identified trends, which are used to determine predefined thresholds of the range of motion constraints using various software tools, including, but are not limited to, 3DCS® Mechanical Modeler, Cortex®, etc.

In step 505, preventive engine 203 of fatigue and injury risk prevention mechanism 105 predicts the fatigue and injury risk to user 101, such as a worker performing material handling operations, using the trained model (trained model of step 402) by comparing the captured motions to the predefined thresholds of the range of motion constraints based on the biomechanical parameters of user 101 from a digital twin model of user 101. For example, preventive engine 203 may predict that user 101 is subject to the risk of fatigue and/or injury risk based on the trained model predicting that user 101 is subject to the risk of fatigue and/or injury risk due to the captured motion (right lateral flexion of lumbar spine at) 25° exceeding a predefined threshold of a range of motion constraint (right lateral flexion of lumbar spine at) 20° involving such captured motion.

As discussed above, in one embodiment, preventive engine 203 uses the trained model (trained model of step 402) which compares the captured motions to predefined thresholds of range of motion constraints based on the biomechanical parameters of user 101 from a digital twin model of user 101. Range of motion constraints, as used herein, refer to the limitations or restrictions placed on the movement of a joint or body part, defining the maximum extent to which it can move in a specific direction, in order to prevent fatigue or injury risk. Biomechanical parameters, as used herein, refer to characteristics relating to the function and motion of the mechanical aspects of users. A digital twin model, as used herein, refers to a digital model of the user that serves as the effectively indistinguishable digital counterpart of the user for practical purposes, such as simulation, testing, and monitoring.

In one embodiment, preventive engine 203 builds a digital twin model of user 101 using various software tools, such as Ansys® Twin Builder, HelixCore, Simio® Simulation, vHive, Veerum, etc. based on real-world data about user 101, which may be acquired from camera-based optical tracking device 102, electromyography sensor 103 and/or inertia measurement unit 104. Such data may include 3D data on the user's body movements during the user's work tasks as well as the associated motion constraints for such body movements. In another embodiment, such data used to create the digital twin model of user 101 is inputted by an expert and/or by user 101.

In one embodiment, the biomechanical parameters of user 101 are acquired from user 101 and/or an expert. Examples of biomechanical parameters include, but are not limited to, height, limb length, and torque limits.

In one embodiment, predefined thresholds for motion constraints, which may be established by experts based on the biomechanical parameters of user 101 from a digital twin model of user 101, are used to determine if user 101 is subject to a risk of fatigue and/or injury risk using the trained model (trained model of step 402).

In one embodiment, predefined thresholds for range of motion constraints are based on the baseline fatigue levels and the baseline motions of user 101.

In step 506, feedback engine 204 of fatigue and injury risk preventive mechanism 105 provides feedback concerning user 101 being subject to the risk of fatigue and/or injury risk based on the prediction of fatigue and injury risk of user 101 performed in step 505.

For example, if the trained model indicates that user 101 is subject to the risk of fatigue and/or injury risk due to the captured motion (right lateral flexion of lumbar spine at) 25° exceeding a predefined threshold of a range of motion constraint (right lateral flexion of lumbar spine at) 20° involving such captured motion, then feedback engine 204 provides feedback indicating that user 101 is subject to the risk of fatigue and/or injury risk. Such feedback may be in the form of video, audio, and/or haptic alerts.

As a result of the foregoing, the principles of the present disclosure provide a means for monitoring users, such as factory workers, and predicting fatigue and injury risk in real-time. Workplace safety and ergonomics may then be improved by detecting potentially harmful motions or excessive fatigue. Providing real-time feedback and fatigue insights significantly reduces lost time, worker compensation claims, and costs associated with injured workers and enable productivity optimizations. By understanding the physical demands of different roles and tasks, employers can implement job rotations, redesign workflows, adjust schedules, and match employees to tasks better suited to their capabilities.

Furthermore, the non-invasive motion tracking and real-time feedback performed by embodiments of the present disclosure provide advantages over current post hoc methods.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A computer-implemented method for predicting fatigue and injury risk to users, the method comprising:
capturing motions performed by a user;
predicting fatigue and injury risk of said user using a trained machine learning model by comparing said captured motions to predefined thresholds of range of motion constraints based on biomechanical parameters of said user from a digital twin model of said user; and
providing feedback based on said predicted fatigue and injury risk of said user.

2. The method as recited in claim 1, wherein said feedback comprises an indication of fatigue and/or injury risk of said user in response to a captured motion of said captured motions exceeding a predefined threshold of said predefined thresholds of range of motion constraints.

3. The method as recited in claim 1, wherein said motions performed by said user are captured using a camera-based optical tracking device.

4. The method as recited in claim 1, wherein said motions performed by said user are captured using an inertia measurement unit.

5. The method as recited in claim 1, wherein said motions performed by said user are captured by an electromyography sensor worn by said user.

6. The method as recited in claim 1, wherein said motions comprise lifting, carrying, and manipulating objects.

7. The method as recited in claim 1, wherein said biomechanical parameters comprise height, limb length, and torque limits.

8. The method as recited in claim 1, wherein said comparison is performed using said machine learning model trained by a machine learning algorithm.

9. The method as recited in claim 8, wherein said machine learning algorithm comprises a long-short-term memory recurrent neural network.

10. The method as recited in claim 1, wherein said predefined thresholds of range of motion constraints are based on baseline fatigue levels and baseline motions of said user.

11. The method as recited in claim 10 further comprising:
saving said captured motions;
identifying trends from said saved captured motions; and
modifying said baseline fatigue levels and said baseline motions of said user based on said identified trends.

12. The method as recited in claim 1, wherein said feedback comprises one or more of the following: visual, audio, and haptic alerts.

13. A computer program product for predicting fatigue and injury risk to users, the computer program product comprising one or more computer readable storage mediums having program code embodied therewith, the program code comprising programming instructions for:
capturing motions performed by a user;
predicting fatigue and injury risk of said user using a trained machine learning model by comparing said captured motions to predefined thresholds of range of motion constraints based on biomechanical parameters of said user from a digital twin model of said user; and
providing feedback based on said predicted fatigue and injury risk of said user.

14. The computer program product as recited in claim 13, wherein said feedback comprises an indication of fatigue and/or injury risk of said user in response to a captured motion of said captured motions exceeding a predefined threshold of said predefined thresholds of range of motion constraints.

15. The computer program product as recited in claim 13, wherein said motions performed by said user are captured using a camera-based optical tracking device.

16. The computer program product as recited in claim 13, wherein said motions performed by said user are captured using an inertia measurement unit.

17. The computer program product as recited in claim 13, wherein said motions performed by said user are captured by an electromyography sensor worn by said user.

18. The computer program product as recited in claim 13, wherein said motions comprise lifting, carrying, and manipulating objects.

19. The computer program product as recited in claim 13, wherein said biomechanical parameters comprise height, limb length, and torque limits.

20. The computer program product as recited in claim 13, wherein said comparison is performed using said machine learning model trained by a machine learning algorithm.

21. The computer program product as recited in claim 20, wherein said machine learning algorithm comprises a long-short-term memory recurrent neural network.

22. The computer program product as recited in claim 13, wherein said predefined thresholds of range of motion constraints are based on baseline fatigue levels and baseline motions of said user.

23. The computer program product as recited in claim 22, wherein the program code further comprises the programming instructions for:
saving said captured motions;
identifying trends from said saved captured motions; and
modifying said baseline fatigue levels and said baseline motions of said user based on said identified trends.

24. The computer program product as recited in claim 13, wherein said feedback comprises one or more of the following: visual, audio, and haptic alerts.

25. A system, comprising:
a memory for storing a computer program for predicting fatigue and injury risk to users; and
a processor connected to said memory, wherein said processor is configured to execute program instructions of the computer program comprising:
capturing motions performed by a user;
predicting fatigue and injury risk of said user using a trained machine learning model by comparing said captured motions to predefined thresholds of range of motion constraints based on biomechanical parameters of said user from a digital twin model of said user; and
providing feedback based on said predicted fatigue and injury risk of said user.

26. The system as recited in claim 25, wherein said feedback comprises an indication of fatigue and/or injury risk of said user in response to a captured motion of said captured motions exceeding a predefined threshold of said predefined thresholds of range of motion constraints.

27. The system as recited in claim 25, wherein said motions performed by said user are captured using a camera-based optical tracking device.

28. The system as recited in claim 25, wherein said motions performed by said user are captured using an inertia measurement unit.

29. The system as recited in claim 25, wherein said motions performed by said user are captured by an electromyography sensor worn by said user.

30. The system as recited in claim 25, wherein said motions comprise lifting, carrying, and manipulating objects.

31. The system as recited in claim 25, wherein said biomechanical parameters comprise height, limb length, and torque limits.

32. The system as recited in claim 25, wherein said comparison is performed using said machine learning model trained by a machine learning algorithm.

33. The system as recited in claim 32, wherein said machine learning algorithm comprises a long-short-term memory recurrent neural network.

34. The system as recited in claim 25, wherein said predefined thresholds of range of motion constraints are based on baseline fatigue levels and baseline motions of said user.

35. The system as recited in claim 34, wherein the program instructions of the computer program further comprise:
saving said captured motions;
identifying trends from said saved captured motions; and
modifying said baseline fatigue levels and said baseline motions of said user based on said identified trends.

36. The system as recited in claim 25, wherein said feedback comprises one or more of the following: visual, audio, and haptic alerts.
